(19) United States
(12) Patent Application Publication
(10) Pub. No.: US 2025/0272497 A1
(43) Pub. Date: Aug. 28, 2025

(54) SYSTEM AND METHOD FOR AI-ASSISTED RESPONSES TO USER QUERIES

(71) Applicant: The Pamoja Institute for Community Engagement and Action, Scarborough (CA)

(72) Inventor: Amanda OTTLEY, Scarborough (CA)

(21) Appl. No.: 19/042,988

(22) Filed: Jan. 31, 2025

Related U.S. Application Data
(60) Provisional application No. 63/558,590, filed on Feb. 27, 2024.

(51) Int. Cl. G06F 40/30; G06F 16/635; G06F 16/638; G16H 80/00

(52) U.S. Cl. CPC G06F 40/30; G06F 16/635; G06F 16/638; G16H 80/00

(57) ABSTRACT

Systems and methods for providing culturally and/or ethnically suitable responses to user queries. An AI-powered system uses a database of prerecorded audio responses that have speech patterns that are specific to any of a specific geographic region, a specific culture, or a specific ethnic group. The system also analyzes the user's audio input for indicators of the user's mental state and takes this into account when formulating a response to the user's query. The audio response is selected, arranged, and stitched together from the various prerecorded audio responses to result in a single audio response that is appropriate to the user and the user's circumstances.

### RELATED APPLICATIONS

This application claims the benefit of U.S. Provisional Application No. 63/558,590 filed on Feb. 27, 2024.

### TECHNICAL FIELD

The present invention relates to cultural and ethnic sensitivity as applied to artificial intelligence systems. More specifically, the present invention relates to systems and methods that provide responses to user queries that are suitable for a specific ethnic and/or cultural context.

### BACKGROUND

In today's world, AI-powered systems are increasingly used to share information, especially health information. Such systems have clear advantages—they can reduce the workload for healthcare professionals and make health-related information more accessible to patients.

However, a significant issue remains: these digital interactions lack the human touch and, in many cases are either inaccessible or at least not inclusive of the different segments of society. Current AI powered systems provide information, but they do so in a sterile, colorless, and nuance-less manner. While such approaches to information dissemination may work for some people and for some portions of society, this one-size-fits-all approach tends to leave other segments isolated. As an example, in some cultures, a nuanced approach or a storytelling-type approach to information dissemination is more effective. Similarly, in other cultures or communities, government or authority-type figures or institutions are untrusted while community figures or family are much more trusted.

Other communities and cultures may not be amenable to a straight information provision approach. A conversational, natural speech approach where the audience absorbs information provided almost passively (or almost in spite of themselves) may, for some contexts, be a much more effective means for information provision.

As well, providing information in a straightforward, nuance free approach using what may not be a mother tongue to some may not be very effective when dealing with those that may be considered as marginalized communities. For such communities, providing the information in a language more accessible to the community and in a form that is more acceptable to the members of the community may be more effective.

Based on the above, there a need for systems and methods that take into account cultural, ethnic, and community standards and needs for better information dissemination.

### SUMMARY

The present invention provides systems and methods for providing culturally and/or ethnically suitable responses to user queries. An AI-powered system uses a database of prerecorded audio responses that have speech patterns that are specific to any of a specific geographic region, a specific culture, or a specific ethnic group. The system also analyzes the user's audio input for indicators of the user's mental state and takes this into account when formulating a response to the user's query. The audio response is selected, arranged, and stitched together from the various prerecorded audio responses to result in a single audio response that is appropriate to the user and the user's circumstances.

In a first aspect, the present invention provides a system for providing audio responses to audio queries, the system comprising:
an input module for receiving an audio input from a user;
a voice analysis module for analyzing said audio input for indicators of a mental state of said user, said voice analysis module producing state data indicative of said possible mental state based on an analysis of said audio input;
an NLP module for analyzing said audio input for indicators regarding a query contained in said audio input, said NLP module producing query data indicative of said query;
a database of prerecorded audio responses;
a trained model module receiving said query data and said state data, said trained model module selecting multiple of said prerecorded audio responses based on said query data and said state data;
a response module receiving prerecorded audio responses selected by said trained model module, said response module arranging and adjusting said selected audio responses to produce a final audio response based on said selected audio responses such that said final audio response approximates regular human speech;
wherein
said prerecorded audio responses include:
   short segments of audio that are interjections;
   long response segments of audio that are responses to specific queries; and
   short segments of audio that are expository in nature;
said regular human speech has speech patterns specific to at least one of:
   a specific geographic region;
   a specific culture; and
   a specific ethnic group.

In a second aspect, the present invention provides a method for providing responses to user queries, the method comprising:
a) receiving user input;
b) analyzing user input using a trained AI-based NLP based model to determine a user query in said user input;
c) producing query data relating to said user query based on results of step b);
d) analyzing query data using a trained AI-based model to determine response data, said response data being suitable for said user query;
e) based on said response data, selecting one or more prerecorded pieces of spoken audio, at least one of said one or more pieces of spoken audio being related to said user query;
f) arranging and adjusting said one or more prerecorded pieces of spoken audio to result in an audio response that, when played, approximates regular human speech;
g) providing said audio response to said user such that said user hears said audio response;
wherein
said regular human speech has speech patterns specific to at least one of:
   a specific geographic region;
   a specific culture; and
   a specific ethnic group.

In a further aspect, the user input may be spoken audio or textual input.

For yet a further aspect of the present invention, the step of analyzing the query data may include analyzing query data for other user inputs from the same user. Similarly, the production of the response data may be based on the query data for the current user input and may be based on query data from previous user inputs from the same user.

A further aspect of the present invention provides that the user input is audio input, and this audio input is analyzed by a voice analysis module for indicators of a possible mental state of the user. The voice analysis module produces state data that is indicative of the possible mental state of the user based on the analysis of the audio input.

### BRIEF DESCRIPTION OF THE DRAWINGS

The embodiments of the present invention will now be described by reference to the following figures, in which identical reference numerals in different figures indicate identical elements and in which:

FIG. 1 is a block diagram detailing generalized workings of a system according to one aspect of the present invention; and

FIG. 2 is a block diagram of a system according to one aspect of the present invention.

### DETAILED DESCRIPTION

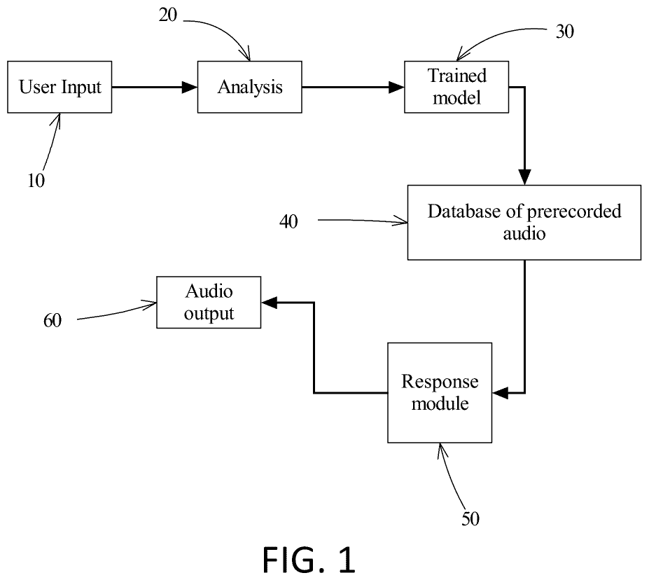

Referring to FIG. 1 a block diagram detailing the general workings of a system according to one aspect of the present invention is illustrated. As can be seen, the system receives user input 10 that is then analyzed 20 to determine the user's query from the user input. Once the query data from the user input has been determined, the query data is then fed into a trained AI-based model 30 to determine response data based on the query data. The response data is related to the user's query and may be directly responsive to the user's query. Based on the response data, one or more prerecorded audio responses are selected from a database 40 of prerecorded audio responses. These selected prerecorded audio responses may be directly responsive to the user's query or, as a whole, may be responsive or at least related to the query. Once the prerecorded audio responses have been automatically selected based on the response data, these selection audio responses are arranged, adjusted, stitched together (using a response module) into a cohesive whole such that the resulting audio mimics or is close to regular human speech.

For clarity, all the prerecorded audio responses are recorded such that they reflect the speech patterns of a specific group such as a specific community, a specific ethnic group, or of a specific culture. This means that the cadence, language, word choice, volume, vocabulary, and even voice is continuously consistent with that specific community, ethnic group, or culture. Thus, depending on the group and the selected approach for information dissemination, the voice may be, for example, that of a community elder, a group leader, or a figure of respect within the group/community (e.g., an elder relative). It should be clear that some of the prerecorded audio responses may be recorded as small/short snippets of audio that can be stitched together to form full responses. Similarly, some of the prerecorded audio responses may be recorded as “filler” or expository material that can help bolster/strengthen a persona that the voice response seeks to portray. As an example, a few short sentences detailing a short anecdote or an expository anecdote may be inserted into the full response to provide the user with a background or “color” as to the persona providing the information (or responding to the user's query).

It should be clear that the prerecorded audio responses may be from a single person such that the user is provided with a unified persona that responds to the user's queries. Different personas may be provided such that different personas (and different genders, ages, backgrounds, etc.) are used to provide information depending on the user's queries. As an example, if a user's query is health related, a nurturing elderly female persona (e.g., a grandmotherly persona) may be used while a user query regarding a legal matter may use an authoritative elderly male persona. Similarly, the choice of persona may also be dependent on the user's circumstances—if the user is determined to be female, the persona used to answer the user's queries may be exclusively female.

It should also be clear that the different prerecorded audio responses, covering the different possible personas and possibly recorded by different people/voice actors, may be stored in a database. The system selects the suitable/appropriate prerecorded audio responses as necessary based on the user circumstances, the user query, and the projected response to the user query.

Once the prerecorded audio responses have been selected for a response to the query, these can then be arranged and/or adjusted in terms of speed, volume, arrangement so that the resulting audio mimics or is close to regular human speech. As noted above, to assist in bolstering a persona for the response, expository snippets may be inserted into the response as well as pauses, nervous audio tics (e.g., coughs, throat clearing, hemming, hawing), and other auditory characteristics that can help “sell” the persona to the user.

While the above notes a user input, this user input may be textual in nature or it may be audio input (i.e., spoken word input). Regardless of the nature of the user input, the system output is an audio response that is presented such that the user can hear the audio response.

For an audio user input, more processing may be performed to provide a suitable response to the user query. While the audio user input may be parsed through a Natural Language Processing (NLP) module to determine the user query from the audio input, the audio input may also be analyzed using a voice analysis module to determine the user's characteristics. As an example, the user's stress levels can be inferred from the audio input, the user's gender can also be inferred, and the cadence, pace, wordiness, pauses, etc. of the user's audio input can be used to determine or infer the user's confidence level, hesitancy, nervousness, and other characteristics. These characteristics can then be used by the various AI enable modules to select a response that not only responds to the query but also to unspoken issues that the user may have. As an example, if the user has a query relating to reproductive matters (e.g., birth control) and the audio input indicates hesitancy, low volume, and a struggle to provide the query, the system may insert snippets into the response that are comforting, nurturing, encouraging while still responding to the query. Accordingly, the system may thus analyze the user's audio input for indicators of the user's mental state and/or user characteristics. These indicators, and the inferred characteristics from these indicators, can then be used by the system, in conjunction with other data, in formulating suitable response data for a response.

It should also be clear that the response to the user query may be based not just on the single user input but on the user's previous queries/inputs. As an example, the user may enter multiple queries in a single session, with the system generating query data and response data for each of the user's queries. These query data and response data, all relating to the same user, may be fed into a trained model module (perhaps also including state data that is indicative of the user's possible mental state and/or characteristics) to result in response data that is suitable for the user's query. The use of previously generated query data, response data, state data for the same user can provide the trained model module with circumstances surrounding the user and the user's query so that a more fulsome and appropriate response can be generated. As noted above, the user's specific query may not be the only issue that the user wants to address but the user's other issues may also need to be addressed.

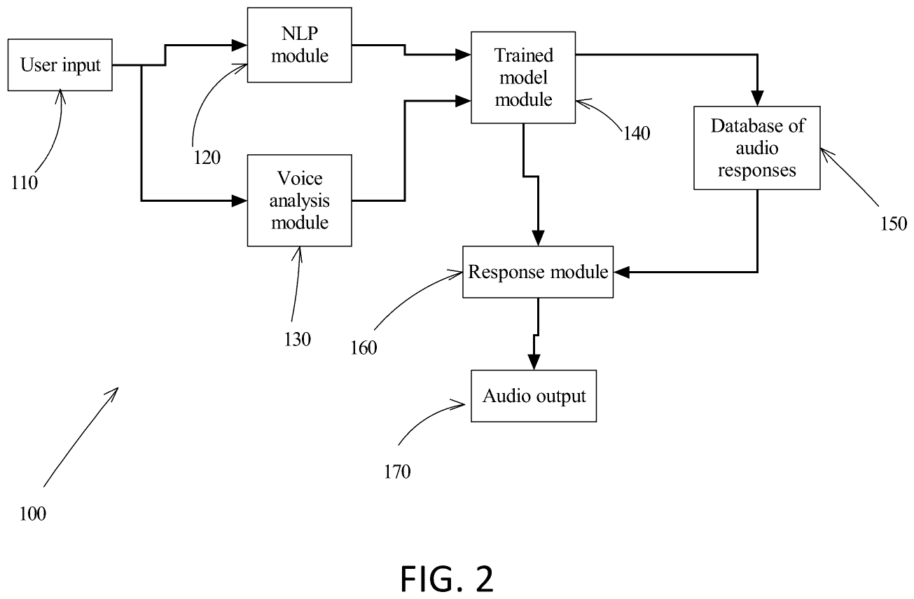

In one aspect, the system of the present invention may be as illustrated in FIG. 2. As can be seen, the system 100 receives user input 110. The user input 110 is received by an NLP module 120 that determines the query data relating to the user query. Simultaneously, the user input 110 may be received by a voice analysis module 130 that analyzes the user audio input 110 for characteristics/indicators of the user's mental state, the user's characteristics, and other indicators that may assist in formulating a suitable response to the user query. The voice analysis module may produce state data indicative of possible user characteristics and/or mental state. This state data and the query data are then received by a trained model module 140 that, based on the input from the different modules, produces response data. This response data is then used as a basis to select one or more prerecorded audio responses from a database 150 of prerecorded audio responses. As noted above, the selected prerecorded audio responses are selected to address the user's query, potentially the user's other unspoken issues, and are selected such that a suitable persona arises from the selections. These selected prerecorded audio responses are then sent to a response module 160 for arrangement/adjustment into a suitable audio response. The selected audio responses are arranged and/or adjusted such that the resulting audio response is a cohesive whole audio that not only responds to the user's query but also to the user's circumstances. The resulting audio response is then provided to the user such that the user hears the response.

As a variant to the above, the trained model module may, in addition to the state data and the query data, receive the state data, query data, and response data from the same user's previous queries. The trained model module may then produce response data based on all these factors and the response data can then be used to select the prerecorded audio responses.

Of course, for clarity, if the user input is textual input, then the voice analysis module 130 is not used and the state data indicating the user's mental state and/or characteristics are not entered into the trained model module 140.

The various modules of the present invention may be implemented using Open AI as well as other AI projects/systems. Similarly, for a better understanding of the present invention, the following reference may be consulted. This reference is hereby incorporated in its entirety by reference.

Weeks R, Sangha P, Cooper L, Sedoc J, White S, Gretz S, et al. Usability and Credibility of a COVID-19 Vaccine Chatbot for Young Adults and Health Workers in the United States: Formative Mixed Methods Study. JMIR Hum Factors. 2023; 30(10): e40533.

It should be clear that the various aspects of the present invention may be implemented as software modules in an overall software system. As such, the present invention may thus take the form of computer executable instructions that, when executed, implements various software modules with predefined functions.

Additionally, it should be clear that, unless otherwise specified, any references herein to ‘image’ or to ‘images’ refer to a digital image or to digital images, comprising pixels or picture cells. Likewise, any references to an ‘audio file’ or to ‘audio files’ refer to digital audio files, unless otherwise specified. ‘Video’, ‘video files’, ‘data objects’, ‘data files’ and all other such terms should be taken to mean digital files and/or data objects, unless otherwise specified.

The embodiments of the invention may be executed by a computer processor or similar device programmed in the manner of method steps or may be executed by an electronic system which is provided with means for executing these steps. Similarly, an electronic memory means such as computer diskettes, CD-ROMs, Random Access Memory (RAM), Read Only Memory (ROM) or similar computer software storage media known in the art, may be programmed to execute such method steps. As well, electronic signals representing these method steps may also be transmitted via a communication network.

Embodiments of the invention may be implemented in any conventional computer programming language. For example, preferred embodiments may be implemented in a procedural programming language (e.g., “C” or “Go”) or an object-oriented language (e.g., “C++”, “java”, “PHP”, “PYTHON” or “C#”). Alternative embodiments of the invention may be implemented as pre-programmed hardware elements, other related components, or as a combination of hardware and software components.

Embodiments can be implemented as a computer program product for use with a computer system. Such implementations may include a series of computer instructions fixed either on a tangible medium, such as a computer readable medium (e.g., a diskette, CD-ROM, ROM, or fixed disk) or transmittable to a computer system, via a modem or other interface device, such as a communications adapter connected to a network over a medium. The medium may be either a tangible medium (e.g., optical or electrical communications lines) or a medium implemented with wireless techniques (e.g., microwave, infrared or other transmission techniques). The series of computer instructions embodies all or part of the functionality previously described herein. Those skilled in the art should appreciate that such computer instructions can be written in a number of programming languages for use with many computer architectures or operating systems. Furthermore, such instructions may be stored in any memory device, such as semiconductor, magnetic, optical or other memory devices, and may be transmitted using any communications technology, such as optical, infrared, microwave, or other transmission technologies. It is expected that such a computer program product may be distributed as a removable medium with accompanying printed or electronic documentation (e.g., shrink-wrapped software), preloaded with a computer system (e.g., on system ROM or fixed disk), or distributed from a server over a network (e.g., the Internet or World Wide Web). Of course, some embodiments of the invention may be implemented as a combination of both software (e.g., a computer program product) and hardware. Still other embodiments of the invention may be implemented as entirely hardware, or entirely software (e.g., a computer program product).

A person understanding this invention may now conceive of alternative structures and embodiments or variations of the above all of which are intended to fall within the scope of the invention as defined in the claims that follow.

We claim:

1. A system for providing audio responses to audio queries, the system comprising:
an input module for receiving an audio input from a user;
a voice analysis module for analyzing said audio input for indicators of a mental state of said user, said voice analysis module producing state data indicative of said possible mental state based on an analysis of said audio input;
an NLP module for analyzing said audio input for indicators regarding a query contained in said audio input, said NLP module producing query data indicative of said query;
a database of prerecorded audio responses;
a trained model module receiving said query data and said state data, said trained model module selecting multiple of said prerecorded audio responses based on said query data and said state data;
a response module receiving prerecorded audio responses selected by said trained model module, said response module arranging and adjusting said selected audio responses to produce a final audio response based on said selected audio responses such that said final audio response approximates regular human speech;
wherein
said prerecorded audio responses include:
short segments of audio that are interjections;
long response segments of audio that are responses to specific queries; and
short segments of audio that are expository in nature;
said regular human speech has speech patterns specific to at least one of:
a specific geographic region;
a specific culture; and
a specific ethnic group.

2. A method for providing responses to user queries, the method comprising:
a) receiving user input;
b) analyzing user input using a trained AI-based NLP based model to determine a user query in said user input;
c) producing query data relating to said user query based on results of step b);
d) analyzing query data using a trained AI-based model to determine response data, said response data being suitable for said user query;
e) based on said response data, selecting one or more prerecorded pieces of spoken audio, at least one of said one or more pieces of spoken audio being related to said user query;
f) arranging and adjusting said one or more prerecorded pieces of spoken audio to result in an audio response that, when played, approximates regular human speech;
g) providing said audio response to said user such that said user hears said audio response;
wherein
said regular human speech has speech patterns specific to at least one of:
a specific geographic region;
a specific culture; and
a specific ethnic group.

3. The method according to claim 2, wherein said user input is spoken audio.

4. The method according to claim 2, wherein said user input is textual input.

5. The method according to claim 2, wherein step d) includes analyzing query data for other user inputs from a same user and producing response data based on query data for a current user input and based on query data from previous user inputs from said same user.

6. The method according to claim 5, wherein said response data determined in step d) is also based on previous response data generated for said previous user inputs from said same user.

7. The method according to claim 3, wherein prior to step c), said user input is analyzed by a voice analysis module for indicators of a possible mental state of said user, said voice analysis module producing state data indicative of said possible mental state based on an analysis of said spoken audio.

8. The method according to claim 7, wherein step d) includes analyzing said state data with said query data to produce said response data.

9. The method according to claim 8, wherein for step e), at least one of said one or more pieces of spoken audio is related to said possible mental state.

10. The system according to claim 1, wherein said system executes a method for providing responses to user queries, the method comprising:
a) receiving user input;
b) analyzing user input using a trained AI-based NLP based model to determine a user query in said user input;
c) producing query data relating to said user query based on results of step b);
d) analyzing query data using a trained AI-based model to determine response data, said response data being suitable for said user query;
e) based on said response data, selecting one or more prerecorded pieces of spoken audio, at least one of said one or more pieces of spoken audio being related to said user query;
f) arranging and adjusting said one or more prerecorded pieces of spoken audio to result in an audio response that, when played, approximates regular human speech;
g) providing said audio response to said user such that said user hears said audio response.
